(19) United States
(12) Patent Application Publication
(10) Pub. No.: US 2008/0262079 A1
(43) Pub. Date: Oct. 23, 2008

(54) Cannabinoid Compositions and Methods of Use Thereof

(76) Inventors: Bernard Mach, Chambesy (CH); Francois Mach, Vesenaz (CH)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/659,751

(22) PCT Filed: Aug. 9, 2005
(86) PCT No.: PCT/IB05/03262

§ 371 Date: Jan. 16, 2008

Related U.S. Application Data
(60) Provisional application No. 60/600,026, filed on Aug. 9, 2004.

(51) Int. Cl. A61K 31/352; A61P 37/00; C12N 5/00; A61P 29/00

(52) U.S. Cl. 514/454; 435/375

(57) ABSTRACT

The present invention provides cannabinoid compositions and the use of such cannabinoid compositions in the treatment of autoimmune and/or inflammatory diseases and disorders. For example, the present invention provides a method of using tetrahydrocannabinoid (THC) compounds in the treatment of autoimmune and/or inflammatory diseases and disorders.

### FIELD OF THE INVENTION

This invention relates generally to the use of cannabinoid compositions in the treatment of autoimmune and/or inflammatory disorders.

### BACKGROUND OF THE INVENTION

Inflammatory disorders include a large number of diseases characterized by the reaction of living tissues to injury, infection or irritation. Autoimmune (immune-mediated) diseases include a large number of diseases characterized by abnormal functioning of the immune system that causes a subject's immune system to produce antibodies against its own tissue. Many vascular disorders, including atherosclerotic forms of such disorders, have an autoimmune component.

Atherosclerosis is a cardiovascular condition occurring as a result of a narrowing of the arterial walls. The narrowing is due to the formation of plaques (raised patches) or streaks in the inner lining of the arteries. These plaques consist of foam cells of low-density lipoproteins, oxidized-LDL, decaying muscle cells, fibrous tissue, clumps of blood platelets, cholesterol, and sometimes calcium. Plaques tend to form in regions of turbulent blood flow and are found most often in people with high concentrations of cholesterol in the bloodstream. The number and thickness of plaques increase with age, causing loss of the smooth lining of the blood vessels and encouraging the formation of thrombi (blood clots). In some instances, fragments of thrombi break off and form emboli, which travel through the bloodstream and block smaller vessels.

The blood supply is restricted to the heart, eventually forming a blood clot leading to death. The major causes of atherosclerosis are hypercholesterolemia and hyperlipidemia is high circulating cholesterol and high lipids like LDL-cholesterol and triglycerides in the blood. These lipids are deposited in the arterial walls, obstructing the blood flow and forming atherosclerotic plaques leading to death.

Atherosclerosis is responsible for more deaths in the U.S. than any other single condition. Atherosclerotic heart disease involving the coronary arteries is the most common single cause of death, accounting for one third of all deaths. Atherosclerotic interference with blood supply to the brain (causing stroke) is the third most common cause of death after cancer. Atherosclerosis also causes a great deal of serious illness by reducing the blood flow in other major arteries, such as those to the kidneys, the legs and the intestines.

Accordingly, there exists a need for novel treatment methods of autoimmune and/or inflammatory disorders such as atherosclerosis.

### SUMMARY OF THE INVENTION

The present invention provides cannabinoid compositions and the use of such cannabinoid compositions in the treatment of autoimmune and/or inflammatory diseases and disorders. For example, the present invention provides a method of using tetrahydrocannabinoid (THC) compounds in the treatment of autoimmune and/or inflammatory diseases and disorders.

The invention provides methods of alleviating a symptom of an autoimmune or inflammatory disorder in a patient (e.g., a human) suffering from, or predisposed to developing, the autoimmune or inflammatory disorder, by administering a cannabinoid composition that contains a cannabinoid or cannabinoid derivative and a pharmaceutically acceptable carrier. The cannabinoid is, for example, delta-9-tetrahydrocannabinoid. The cannabinoid or cannabinoid derivative is administered in a non-psychotropic dosage for the patient. The autoimmune or inflammatory is, for example, atherosclerosis. Other autoimmune or inflammatory disorders include, but are not limited to autoimmune uveitis, atopic dermatitis, vasculitis, psoriasis, ulcerative colitis, Crohn's disease, myositis, vitiligo, type I diabetes, thyroidites (hashimoto), juvenile arthritis, contact dermatitis, lupus erythematosus, and myastenia gravis.

Methods of the invention include a method of alleviating a symptom of atherosclerosis in a patient suffering from, or predisposed to developing, atherosclerosis, by administering a cannabinoid composition that contains delta-9-tetrahydrocannabinoid and a pharmaceutically acceptable carrier. Delta-9-tetrahydrocannabinoid is administered, for example, in a non-psychotropic dosage.

The invention also provides methods of activating a regulatory T-lymphocyte by contacting the regulatory T-lymphocyte with a cannabinoid composition that contains a cannabinoid or cannabinoid derivative and a pharmaceutically acceptable carrier. The cannabinoid is, for example, delta-9-tetrahydrocannabinoid. The regulatory T-lymphocyte is contacted, for example, in vivo, in vitro, or ex vivo.

Methods of the invention also include methods of decreasing cellular proliferation by contacting a cell with a cannabinoid composition that includes a cannabinoid or cannabinoid derivative and a pharmaceutically acceptable carrier. The cannabinoid is, for example, delta-9-tetrahydrocannabinoid. The cell is contacted, for example, in vivo, in vitro, or ex vivo. Suitable cells for use with these methods include, for example, splenocytes and lymph node cells.

The invention also provides methods of decreasing cytokine production by contacting a cell with a cannabinoid composition that includes a cannabinoid or cannabinoid derivative and a pharmaceutically acceptable carrier. . The cannabinoid is, for example, delta-9-tetrahydrocannabinoid. The cell is contacted, for example, in vivo, in vitro, or ex vivo. Suitable cells for use with these methods include, for example, splenocytes and lymph node cells. Cytokines include e.g., interferon-gamma (IFN-γ), IL-10, and transforming growth factor-beta (TGF-β).

### BRIEF DESCRIPTION OF THE DRAWINGS

FIGS. 1A-1H are a series of photographs depicting the expression of cannabinoid receptor CB2 in human and mouse atherosclerotic plaques. FIG. 1A-1H depict representative cryosections of human coronary atherosclerotic lesion (Panels A and B), mouse aortic arch lesion (Panels C and D), mouse aortic root lesion (Panels E and F), and mouse spleen for control (Panels G and H). The sections were immunostained with anti-CB2 receptor (Panels A, C, E, G) or with secondary antibody only (Panels B, D, F, and H).

FIGS. 2A-2C are a graph and a series of photographs depicting reduced atherosclerotic plaque development in THC-treated apoE^{-/-} mice after 11 weeks of high cholesterol diet. FIGS. 2B and 2C depict representative cryosections of mice aortic roots, stained for lipid deposition with Sudan IV at 5 weeks (Panel 2B) and at 11 weeks (Panel 2C) of feeding with high cholesterol diet, and Panel 2A is a graph depicting the quantification of the atherosclerotic lesions in each aortic root. The induction of atherogenesis in apoE^{-/-} mice after 5 weeks of feeding with a high cholesterol diet was compared to mice under normal diet. THC (1 mg/kg) was administered daily and orally during the last 6 weeks of the 11 week diet group. *=p<0.05 vs. wild-type; **=p<0.05 vs. apoE^{-/-} 5 weeks; ***=p<0.05 vs. apoE^{-/-} mice 11 weeks without THC.

FIGS. 3A-3E are a series of graphs depicting the reduction of proliferative response and the inhibition of Th1 polarization in the presence of THC. In Panel A, isolated splenocytes from THC-treated or control apoE^{-/-} mice under high cholesterol diet were stimulated with conA for 72 hours, and the proliferation rate was determined using the non-radioactive MTS colorimetric assay. The amount of 490 nm absorbance is directly proportional to the number of living cells. In Panels B, C and D, isolated splenocytes from THC-treated or control apoE^{-/-} mice were stimulated with conA for 48 to 72 hours, and the concentrations of IFN-γ, IL-10 and TGF-β were determined by ELISA in the culture supernatants. Panel E depicts Th1/Th2 cytokine ratio (ratio of IFN-γ to IL-10 concentration). The data represent mean values±SEM; *=p<0.05.

FIG. 4 is a graph depicting the reduced migration of peritoneal macrophages in vitro in the presence of THC. Thioglycollate-elicited peritoneal cavity macrophages obtained from apoE^{-/-} mice were analyzed for their ex vivo migration capacity by chemo-attraction to MCP-1. The data represent mean values±SEM; *=p<0.05 for both unstimulated (w/o) and stimulated THC-treated cells vs. IFN-γ stimulated cells.

### DETAILED DESCRIPTION

The present invention provides cannabinoid compositions and the use of such cannabinoid compositions in the treatment of autoimmune and/or inflammatory diseases and disorders. For example, the present invention provides a method of using tetrahydrocannabinoid (THC) compounds in the treatment of autoimmune and/or inflammatory diseases and disorders. A cannabinoid composition includes a cannabinoid (e.g., delta-9-tetrahydrocannabinoid), or a cannabinoid derivative, and a pharmaceutically acceptable carrier. “Pharmaceutically Acceptable Carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, the nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients. The term patient includes human and veterinary subjects. Cannabinoid Compounds Cannabis sativa, commonly known as marijuana, has been used for several years for its medicinal effects, including antipyretic and analgesic properties. Approximately 80 cannabis constituents, termed cannabinoids, naturally occur as 21 carbon atom compounds of cannabis and analogues of such compounds and their metabolites.

One cannabinoid is delta-9-tetrahydrocannabinol (A₉-THC or delta-9-THC), the major physiologically active constituent of marijuana. Other cannabinoids and cannabinoid derivatives include, for example, cannabidiol (CBD), cannabinol (CBN), tetrahydrocannbidiol acid, nabilone (a delta-9-THC intermediate), and any of a variety of synthetic derivatives known in the art.

Delta-9-THC, the major psychoactive component of marijuana, is a controlled substance because it has both sedative and depressant-like effects on the cardiovascular and central nervous systems, as opposed to cannabidiol, a non-psychoactive constituent of marijuana. Delta-9-tetrahydrocannabinol is currently approved by regulatory authorities for use as an antiemetic in cancer chemotherapy as well as an appetite stimulant for patients inflicted with the AIDS virus.

The pharmacokinetics of THC varies with the route of administration. When smoked, Delta 9-THC is rapidly absorbed by the blood in the lungs. Oral absorption of THC is less rapid than from the lungs. One difference between smoking and ingestion as means of THC administration is that when cannabinoids are absorbed from the gut, the blood containing them first goes directly through the liver. The liver rapidly clears the Delta 9-THC from the blood and enzymatically changes much of the Delta 9-THC to other metabolites before much of the Delta 9-THC can reach the brain. For example, a large proportion is metabolized to 11-hydroxy delta 9-THC. When taken orally, two to three times more Delta 9-THC is required to obtain equivalent acute psychological and physiological effects, as compared with THC administered by smoking. Thus, the relatively low oral dosages of THC used the methods of the invention are non-psychotropic dosages.

The structure of delta-9-THC is shown below in Formula 1:

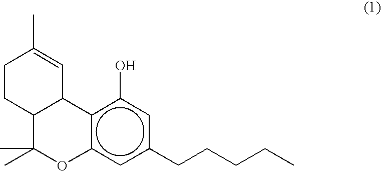

### Cannabinoid Compositions and the Treatment of Autoimmune and Inflammatory Disorders

The identification and development of potential promising novel anti-inflammatory therapies is of great medical interest. Immunosuppressive and anti-inflammatory effects of cannabinoids have been reported (see e.g., Srivastava, Immunopharmacology 40, 179-85 (1998); Zhu, J Immunol 165, 373-80 (2000); and Yuan, J Neuroimmunol 133, 124-31 (2002)), and pre-clinical studies provided the therapeutic rationale for use in treating autoimmune diseases such as multiple sclerosis (Lyman, J Neuroimmunol 23, 73-81 (1989)) or rheumatoid arthritis (Malfait, Proc Natl Acad Sci USA 97, 9561-6 (2000)). In murine collagen-induced arthritis, a mouse model of rheumatoid arthritis, cannabidiol, a major cannabinoid derivative, ameliorated chronic inflammation by inhibiting Th1 responses, as shown by reduced proliferation and IFN-γ production of lymph node cells from treated mice. (Malfait, (2000)).

In support of the immunomodulatory role of cannabinoids, receptors for THC have been identified on several types of immune cells. While the CB1 receptor is expressed predominantly in the brain, the CB2 receptor expression is found primarily on cells of the immune system, such as B cells, T cells, and monocytes. (Klein, J Leukoc Biol 74, 486-96 (2003)). It has been suggested that the immunomodulatory effects of cannabinoids are mediated by the CB2 receptor expressed on immune cells. (Buckley, Eur J Pharmacol 396, 141-9 (2000)). The fact that THC-mediated inhibition of helper T cell activation is not observed in CB2 receptor^{-/-} mice, strongly supports this hypothesis. (Buckley (2000)). These immunomodulatory properties suggest that cannabinoid derivatives are beneficial in the treatment of atherosclerosis.

Atherosclerosis is a chronic inflammatory disease of the large arteries that represents the primary cause for heart disease and stroke. (Libby, Nature 420, 868-74 (2002)). It is now generally recognized that atherosclerosis is a chronic inflammatory disease that can lead to acute clinical events following plaque rupture and thrombosis. (Libby, Nature 420:868-74 (2002); Libby, Circulation 105, 1135-43 (2002)). Prevention and current treatments for atherosclerosis are mainly based on drugs that lower plasma cholesterol concentration and high blood pressure. In particular, statins have proven to reduce cardiovascular events significantly, not only by their cholesterol-lowering properties, but also by their more recently identified anti-inflammatory and immunomodulatory effects (Mach, Circulation 109, II15-7, (2004)). Nevertheless, atherosclerosis remains the primary cause of heart disease and stroke accounting for up to 50% of all deaths in Western countries.

As described above, derivatives of cannabinoids such as delta-9-Tetrahydrocannabinol (THC) modulate immune functions (Klein, J Leukoc Biol 74, 486-96 (2003)), and therefore, the derivatives have a therapeutic potential for the treatment of inflammatory diseases. In the studies described in the Examples presented herein, the effect of THC on established (e.g., clinically manifested) atherosclerosis in a murine model was evaluated. As described herein, oral administration of THC (1 mg/kg per day) resulted in the significant inhibition of disease progression, as demonstrated by reduced atherosclerotic plaque development within the aortic roots. This effective dose is lower than the dose required for psychotropic effects of THC. Furthermore, CB2 receptor, the main cannabinoid receptor expressed on immune cells (Klein, J Leukoc Biol 74, 486-96 (2003); Buckley, Eur J Pharmacol 396, 141-9 (2000)), was detected in both human and mouse atherosclerotic plaques. Lymphoid cells isolated from THC-treated mice exhibited diminished proliferation capacity as well as IFN-γ secretion, whereas IL-10 and TGF-β production were not significantly altered. In vitro, THC also inhibited macrophage chemotaxis, a crucial step for the development of atherosclerosis (Libby, Nature 420, 868-74 (2002)). The data described herein demonstrate that oral treatment with a low dose of THC, through its plieotropic immunomodulatory effects on lymphoid cells and monocytes, is a potent inhibitor of atherosclerosis progression in mice. Thus, THC therapy is beneficial for the treatment of patients with clinically manifested atherosclerosis.

Thus, the Examples provided herein have shown that oral THC treatment, provided at a relatively low dose, and initiated after manifestation of clinically detectable artery lesions, significantly inhibits atherosclerosis progression in mice. This anti-atherosclerotic effect is likely mediated by the CB2 receptor, as this receptor is strongly expressed in atherosclerotic lesions. The data presented herein also provide evidence that the anti-atherosclerotic properties of THC are associated with a reduction of Th1 response and an inhibition of monocyte migration to the site of inflammation. These results suggest a therapeutic potential for low doses of cannabinoid derivatives as novel anti-inflammatory agents for patients with clinically manifested atherosclerosis.

Additionally, the cannabinoid compositions of the invention are used in therapeutic formulations for the treatment of an autoimmune disease or an inflammatory disorder. The present invention also provides methods of treating or alleviating a symptom associated with any of the autoimmune diseases and inflammatory disorders described herein.

Autoimmune diseases include, for example, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjögren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders, include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

Symptoms of atherosclerosis include, for example, ischemia, elevated blood pressure, headaches, dizzy spells, muscle aches and/or cramps, fatigue, and pain (e.g., chest pain (angina) when a coronary artery is involved, or leg pain when a leg artery is involved). Symptoms associated with autoimmune diseases include, for example, symptoms associated with uveitis: redness and watering of the eye, sensitivity to bright light, blurry vision, aching in the eye, a small, irregularly shaped pupil, ocular complications (e.g., glaucoma, cataracts or retinal damage); symptoms associated with skin disorders (e.g., dermatitis, psoriasis) such as itchiness or burning in the affected skin areas, localized swelling, and rash; symptoms associated with vasculitis such as malaise, fever, weight loss, fatigue, a rapid pulse and general aches and pains; symptoms associated with intestinal disorders (e.g. ulcerative colitis and Crohn's disease) such as fatigue, weight loss, loss of appetite, rectal bleeding, fever, bloody diarrhea, nausea, severe abdominal cramps, arthritis, inflammation of the eye, liver disease (e.g., hepatitis, cirrhosis, and primary sclerosing cholangitis), osteoporosis, skin rashes, and anemia; symptoms associated with type I diabetes such as exceptional thirst, dry mouth, frequent urination, weight loss, fatigue, and blurry vision; symptoms associated with juvenile arthritis such as persistent joint swelling, pain, stiffness, fever, skin rash, and swollen lymph nodes; and symptoms associated with lupus erythematosus such as achy joints (arthralgia), fever over 100 degrees F. (38 degrees C.), prolonged or extreme fatigue, arthritis (swollen joints), skin rashes, anemia, kidney problems, chest pains, skin rash, photosensitivity.

In one embodiment, the cannabinoid compositions used to treat an autoimmune and/or inflammatory disorder are administered in combination with any of a variety of known anti-inflammatory and/or immunosuppressive compounds. Suitable known compounds include, but are not limited to methotrexate, cyclosporin A (including, for example, cyclosporin microemulsion), tacrolimus, corticosteroids, statins, interferon beta, Remicade (Infliximab), Enbrel (Etanercept) and Humira (Adalimumab).

### Therapeutic Administration and Formulations

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci.89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Since the cannabinoid compounds of the present invention, e.g., THC compounds, are intended for use in pharmaceutical compositions, it will be understood that each is provided in substantially pure form, for example at least 50% pure, preferably more than at least 75% pure and more preferably at least 95% pure (% are on a wt/wt basis).

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples, including the experiments conducted and results achieved are provided for illustrative purposes only and are not to be construed as limiting upon the present invention.

### Example 1

### Effect of Cannabinoid Derivatives on Atherosclerotic Lesions

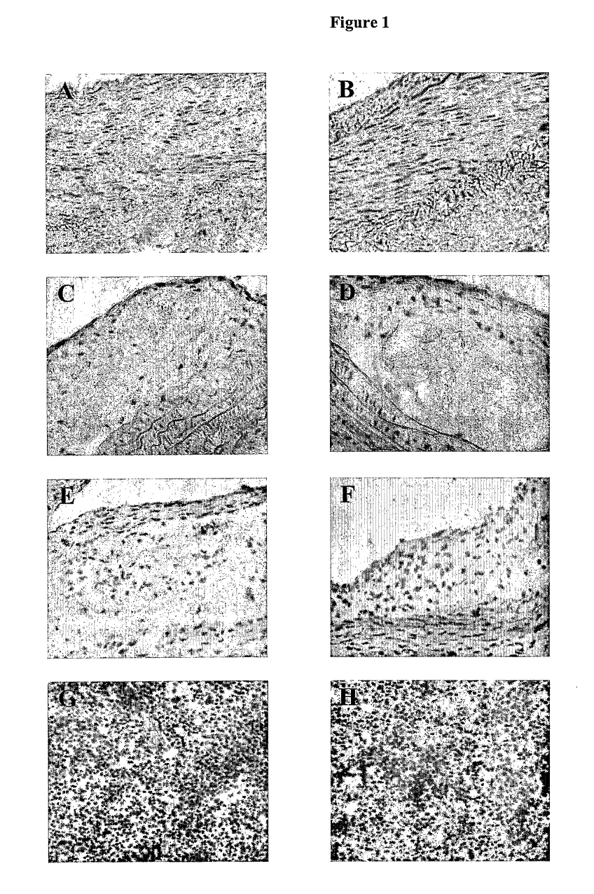

The studies presented herein were based on the hypothesis that cannabinoid treatment would alter inflammatory processes pivotal for the development of atherosclerosis thus, limiting disease progression. The expression of CB2 receptors in atherosclerotic plaques of human and mouse diseased arteries was first evaluated. Immunohistochemistry revealed extensive amounts of CB2 receptor within human coronary atheroma as well as atherosclerotic lesions of mouse aortic arch and root (FIG. 1), while none was observed in non-diseased arteries. CB1 receptors were not detected in any vascular tissue.

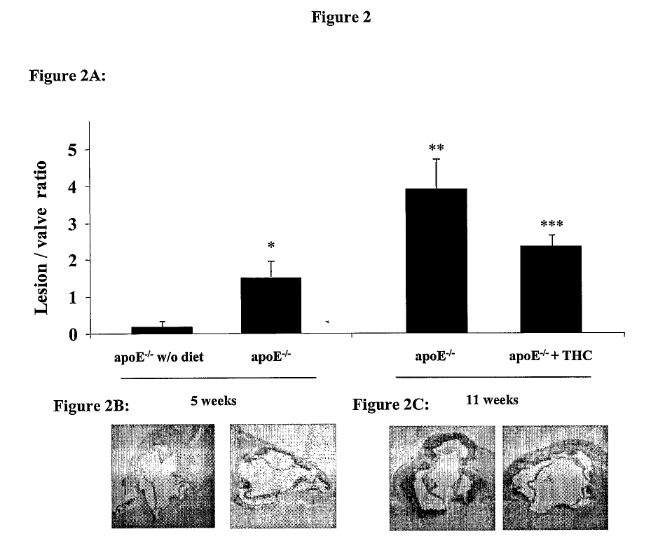

The anti-atherosclerotic potential of THC was tested in the apolipoprotein E^{-/-} (apoE^{-/-}) mouse model of atherosclerosis. The studies presented herein utilized delta-9-Tetrahydrocannoabinol (THC), the major component of marijuana, as its immune-modulating effects have been well-documented in several studies. (See e.g., Srivastava, Immuopharmacology 40, 179-85 (1998); Zhu, J Immunol 165, 373-80 (2000); Yuan J Neuroimmunol 133, 124-31 (2002); Lyman, J Neuroimmunol 23, 73-81 (1989); Malfait, Proc Natl Acad Sci USA 97, 9561-6 (2000)). In addition, this compound is already commercially available, for example as an anti-vomiting drug or for use in treating anorexia. Since potential novel anti-inflammatory therapies should be well tolerated and preferably devoid of psychotropic effects, a low dosage of THC (e.g., 1 mg/kg) was used for daily oral administration. Analysis of THC levels in blood serum of THC-treated mice revealed a concentration of 0.6 ng/ml, which is considered as non-psychotropic in humans. (Chesher, et al., Pharmacol Biochem Behav 35:861-4 (1990); Brenneisen, et al., Int J Clin Pharmacol Ther 34, 446-52 (1996); Chan, et al., Fundam Appl Toxicol 30, 109-17 (1996)) To test the therapeutic effect of THC on established atherosclerosis, apoE^{-/-} mice were fed with a high cholesterol diet for 5 weeks, and THC was then administered during the following 6 weeks, while maintaining the cholesterol diet. After 5 weeks of diet, atherosclerotic lesions were clearly detectable within the aortic roots of apoE^{-/-} mice compared to apoE^{-/-} mice under normal diet (FIG. 2). More advanced vascular lesion development occurred within the aortic roots compared to the abdominal aorta, as demonstrated in previous studies (See e.g., Nakashima, Arterioscler Thromb 14, 133-40 (1994), Reddick, Arterioscler Thromb 14, 141-7 (1994); and Tangirala, J Lipid Res 36, 2320-8 (1995)). After 11 weeks of diet, there was marked progression of atherosclerotic lesions within the aortic roots of control mice, while THC-treated mice exhibited significantly reduced progression of atherosclerotic lesions (FIG. 2). Similar results were observed within the abdominal aorta. No differences occurred in serum cholesterol and triglyceride levels or body weights between the two groups. None of the THC-treated mice died during treatment, and none showed unhealthy behavior.

### Example 2

### Effect of Cannabinoid Derivatives on Cytokine Expression

Recent studies indicated that progression of atherosclerosis may result from an imbalance between pro- and anti-inflammatory mediators in response to endothelial injury. (Daugherty, Circ Res 90, 1039-40 (2002)). Several reports demonstrated that T cells play a major role during early atherosclerosis development. (Song, L., J Clin Invest 108, 251-9 (2001); Moeller, Atherosclerosis 168, 49-56 (2003)). In support of this hypothesis, it has been shown that Th1 cells represent the predominant population of activated T cells within atherosclerotic lesions. (Benagiano, Proc Natl Acad Sci USA 100, 6658-63 (2003); Laurat, Circulation 104:197-202 (2001)). Other experiments demonstrated that treatment with THC regulates the Th1/Th2 balance in activated T cells. (Zhu, J Immunol 165, 373-80 (2000); Yuan, J Neuroimmunol 133, 124-31 (2002)).

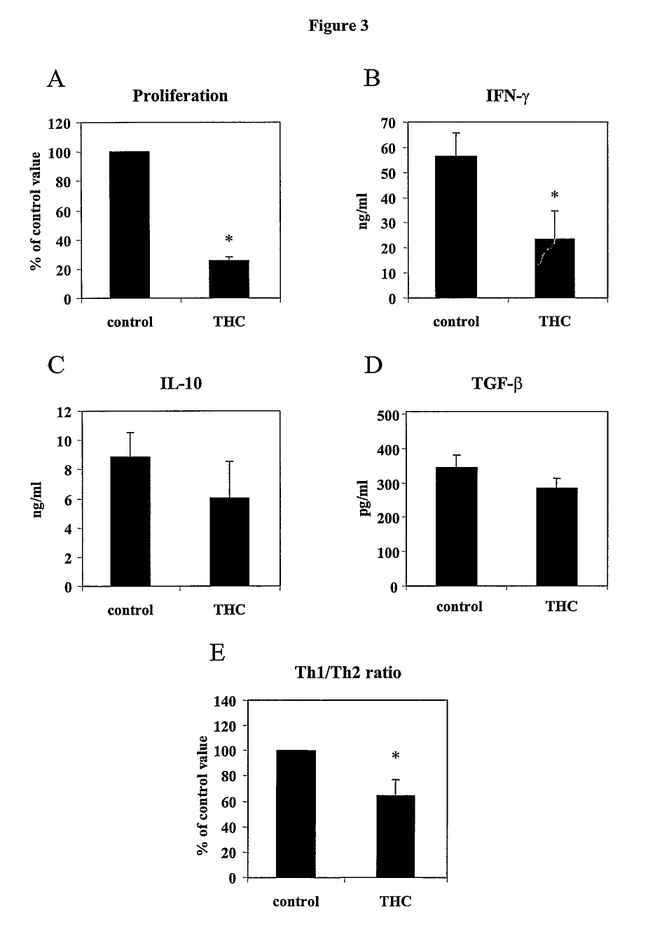

The studies presented herein were based on the hypothesis that the observed anti-atherosclerotic effects of THC might result from a modified cytokine expression pattern in the THC-treated mice. Therefore, the influence of THC treatment on inflammatory responses during the beginning of atherosclerosis development was investigated. Proliferative responses and cytokine profiles of lymphoid cells isolated from mice on a high cholesterol diet with and without administration of THC were analyzed. Compared to untreated mice, treatment with THC significantly reduced proliferative responses of in vitro stimulated splenocytes (FIG. 3A). Similar results were obtained with lymph node cells. Culture supernatants were examined for Th1 (IFN-γ, IL-12) and Th2 (IL-4, IL-10) cytokines, as well as TGF-β. In comparison with untreated mice, THC-treated mice produced significantly less IFN-γ, whereas only a modest, but not significant, downregulation of the Th2 cytokine IL-10 or TGF-β was detectable (FIGS. 3B-3D). In both THC-treated and untreated groups, neither expression of IL-4 nor IL-12 was detectable. Thus, during atherosclerosis, THC seems to exert its anti-inflammatory activity through suppression of the Th1 response, resulting in a shift of the Th1/Th2 balance (FIG. 3E).

### Example 3

### Effect of Cannabinoid Derivatives on Cell Migration

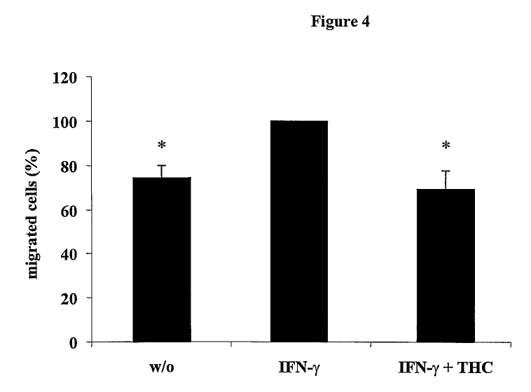

Early processes of atherosclerosis involve endothelial dysfunction in response to cardiovascular risk factors, which triggers the recruitment of leukocytes (monocytes/macrophages and T lymphocytes) into the vessel wall. The studies described herein address whether treatment with THC inhibited cell migration using a functional in vitro experiment. Thioglycollate-stimulated peritoneal mouse macrophages were isolated from apoE^{-/-} mice and assayed for their migration capacity by chemo-attraction to MCP-1. Treatment with THC at a concentration corresponding to the serum levels observed in THC-treated mice (i.e., 0.6 ng/ml) significantly inhibited migration of macrophages (FIG. 4).

### Example 4

### Materials and Methods

Reagents: Synthetic delta-9-THC Marinol® (Dronanbinol, Unimed Pharmaceuticals, Inc.; Marietta Ga., USA) was dissolved at 0.1 mg/ml in 5.5.% fat milk (w/v) in water. THC (1 mg/kg per day) in 1.5% fat milk (w/v) was administered orally within drinking water. Delta-9-THC for in vitro experiments was purchased as a stock solution of 1 mg/ml in methanol (Cambridge Isotype Laboratories, Andover Mass., USA) and were further diluted in warm medium immediately before use. For in vitro transmigration assays, all experiments were performed by adding corresponding dilutions of the THC vehicle (methanol) to the non-THC-treated controls.

Animals: As a model of in vivo atherosclerosis, 10-week old male apolipoprotein E^{-/-} mice (apoE^{-/-}) C57BL/6 mice were used. For histological and atherosclerotic plaque development analysis, mice were fed with a high cholesterol diet (1.25% cholesterol, 0% cholate; Research Diets, New Brunswick N.J., USA) for 5 weeks or for 11 weeks (n=6 per group). THC was administered during the last 6 weeks of the 11 week diet group. In parallel, control mice (littermates) received milk without THC (n =8). For proliferation and cytokine analysis, littermate apoE^{-/-} mice were divided into 2 groups (control, THC; n=6 per group) and fed with a high cholesterol diet for 4 weeks. THC or milk only was administered during the last 2 weeks of diet. All animal studies were approved by the local Ethical Committee.

Histological analysis: Surgical specimens of human coronary atheroma were obtained. Immunostaining of methanol-acetone-fixed cryosections of human and mouse atherosclerotic arteries was performed as previously described (Mulhaupt, Cardiovasc Res., 59(3):755-66 (2003); Kwak et al., Circulation 107, 1033-9 (2003)), using a rabbit polyclonal antibody against CB2 receptor (Cayman Chemical, Ann Arbor Mich., USA).

Atherosclerotic lesion size quantification: Atherosclerotic lesions within the thoraco-abdominal aorta and aortic roots were analyzed by Sudan IV staining for lipid deposition. Quantification was performed by computer image analysis using the MetaMorph6 software (Zeiss, Feldbach, Switzerland) as previously described. (Kwak et al., Circulation 107, 1033-9 (2003)).

Blood Analysis: For measurements of cholesterol and triglyceride content, blood samples were collected at the beginning and the end of the diet. HDL and VLDL cholesterol fractions of sera were measured by fast protein lipid chromatography. THC levels in blood (after 2, 4 and 6 weeks of treatment) were measured by gas chromatography/mass spectrometry with a limit of detection of 0.5 mg/ml as previously described. (Giroud, et al. Forensic Sci Int 123, 159-64 (2001)).

Proliferation Assay: Splenocytes (SC) or lymph node cells (LNC) were isolated from THC- or milk-treated mice and cultured in 96-well plates at a concentration of 5×10⁶ cells/ml. Culture medium consisted of RPMI 1640 supplemented with 25 mM HEPES buffer, 2 mM L-glutamine, 100 U/ml penicillin, 0.1 mg/ml streptomycin, and 10% heat-inactivated fetal bovine serum (FBS). Cells were stimulated in triplicates with varying concentrations of concanavalin (con A; Sigma). After 72 hours, cell proliferation was determined using a non-radioactive MTS cell proliferation assay (Promega) according to the manufacturer's guidelines.

Cytokine analysis: For cytokine analysis, LNC and SC were cultured under the same conditions as described for the proliferation assay and stimulated with 2 micrograms per milliliter (2 μg/ml) conA, and supernatants were recovered after 48 hours (for IFN-γ, IL-12, TGF-β measurement) and 72 hours (for IL-14 and IL-10). Murine IFN-γ, IL-12 (p70), IL-4, IL-10, and TGF-β were assayed by ELISA using paired antibodies according to the manufacturer's instructions (R&D Systems).

Transmigration assay: Macrophages from the peritoneal cavity of thioglycollate-injected apoE^{-/-} mice were isolated 4 days post-injection and stimulated (in triplicates) for 4 hours with 1 ng/ml mIFN-γ in the presence or absence of 0.6 ng/ml THC (n=4 mice). Stimulated cells as well as unstimulated cells (as a control) were transfected into the upper compartment of transwell filter inserts, and chemo-attracted by adding 1 nM MCP-1 (R&D Systems) to the lower transwell compartment. Following 90 minutes of incubation, the number of migrated cells was determined by counting (blind observers) 10 microscopic fields per well.

Statistical analysis: All results are expressed as mean±SEM Differences between the values were considered significant at p<0.05 using the two-tailed Student's T-test.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of alleviating a symptom of an autoimmune or inflammatory disorder in a patient comprising administering a cannabinoid composition comprising a cannabinoid or cannabinoid derivative and a pharmaceutically acceptable carrier, wherein said patient is suffering from, or predisposed to developing, said autoimmune or inflammatory disorder.

2. The method of claim 1, wherein the cannabinoid is delta-9-tetrahydrocannabinoid.

3. The method of claim 1, wherein said cannabinoid composition comprises a non-psychotropic dosage of a cannabinoid or a cannabinoid derivative.

4. The method of claim 1, wherein said autoimmune or said inflammatory disorder is atherosclerosis.

5. The method of claim 1, where said autoimmune or said inflammatory disorder is selected from the group consisting of autoimmune uveitis, atopic dermatitis, vasculitis, psoriasis, ulcerative colitis, Crohn's disease, myositis, vitiligo, type I diabetes, thyroidites (hashimoto), juvenile arthritis, contact dermatitis, lupus erythematosus, and myastenia gravis.

6. The method of claim 1, wherein said patient is a human.

7. A method of alleviating a symptom of atherosclerosis in a patient comprising administering a cannabinoid composition comprising delta-9-tetrahydrocannabinoid and a pharmaceutically acceptable carrier, wherein said patient is suffering from, or predisposed to developing, atherosclerosis.

8. The method of claim 7, wherein said cannabinoid composition comprises a non-psychotropic dosage of delta-9-tetrahydrocannabinoid.

9. A method of activating a regulatory T-lymphocyte comprising contacting said regulatory T-lymphocyte with a cannabinoid composition comprising a cannabinoid or cannabinoid derivative and a pharmaceutically acceptable carrier.

10. The method of claim 9, wherein the cannabinoid is delta-9-tetrahydrocannabinoid.

11. The method of claim 9, wherein said regulatory T-lymphocyte is contacted in vivo.

12. The method of claim 9, wherein said regulatory T-lymphocyte is contacted in vitro.

13. The method of claim 9, wherein said regulatory T-lymphocyte is contacted ex vivo.

14. A method of decreasing cellular proliferation comprising contacting a cell with a cannabinoid composition comprising a cannabinoid or cannabinoid derivative and a pharmaceutically acceptable carrier.

15. The method of claim 14, wherein the cannabinoid is delta-9-tetrahydrocannabinoid.

16. The method of claim 14, wherein said cell is contacted in vivo.

17. The method of claim 14, wherein said cell is contacted in vitro.

18. The method of claim 14, wherein said cell is contacted ex vivo.

19. The method of claim 14, wherein said cell is a splenocyte or a lymph node cell.

20. A method of decreasing cytokine production comprising contacting a cell with a cannabinoid composition comprising a cannabinoid or cannabinoid derivative and a pharmaceutically acceptable carrier.

21. The method of claim 20, wherein the cannabinoid is delta-9-tetrahydrocannabinoid.

22. The method of claim 20, wherein said cell is contacted in vivo.

23. The method of claim 20, wherein said cell is contacted in vitro.

24. The method of claim 20, wherein said cell is contacted ex vivo.

25. The method of claim 20, wherein said cell is a splenocyte or a lymph node cell.

26. The method of claim 20, wherein said cytokine is selected from the group consisting of interferon-gamma (IFN-γ), IL-10, and transforming growth factor-beta (TGF-β).
